(19) United States
(12) Patent Application Publication
(10) Pub. No.: US 2023/0282341 A1
(43) Pub. Date: Sep. 7, 2023

(54) Methods and systems for connecting subjects to healthcare providers

(71) Applicant: Arun Srinivasan, Norton, MA (US)

(72) Inventor: Arun Srinivasan, Norton, MA (US)

(21) Appl. No.: 18/178,513

(22) Filed: Mar. 5, 2023

Related U.S. Application Data
(60) Provisional application No. 63/317,050, filed on Mar. 6, 2022.

(51) Int. Cl. G16H 40/20; G16H 20/00

(52) U.S. Cl. CPC G16H 40/20; G16H 20/00

(57) ABSTRACT

Systems and methods are provided herein for connecting at least one subject to at least one health care provider, the method including: scanning the subject with an exploratory device to obtain an initial exploratory result; sending the initial exploratory result to the at least one health care provider for analysis; and receiving and displaying at least one treatment plan from the at least one health care provider thereby connecting the subject to the health care provider.

### RELATED APPLICATIONS

This application claims benefit of priority from U.S. Provisional application No. 63/317,050 having the title, “Methods and systems for connecting subjects to healthcare providers” by inventor Dr. Arun Srinivasan, filed Mar. 6, 2022. The application is hereby incorporated by reference herein in its entirety.

### BACKGROUND

A subject requiring treatment must find and contact a health care provider to make an appointment before beginning the treatment. In many instances, the subject is not even aware of their health status and that they require treatment. For procedures that are partially covered or not covered by health or dental insurance, the cost for treatment varies from provider to provider. Therefore, the subject must visit multiple providers to find the right provider and cost for their needs.

There is a need for a platform to connect subjects with the right health care providers in which the subject does not have to visit multiple health care providers.

### SUMMARY

An aspect of the invention described herein provides a method for connecting at least one subject to at least one health care provider, the method including: scanning the subject with an exploratory device to obtain an initial exploratory result; sending the initial exploratory result to the at least one health care provider for analysis; and receiving and displaying at least one treatment plan from the at least one health care provider thereby connecting the subject to the health care provider.

An embodiment of the method further includes prior to sending, identifying at least one health condition in the subject from the initial exploratory result. An embodiment of the method further includes after identifying, classifying the subject by severity of the at least one health condition. An embodiment of the method further includes after displaying, the subject selecting at least one treatment plan from the at least one health care provider.

An embodiment of the method further including choosing at least one of the exploratory device from: an x-ray, an intra-oral scanner, a cone beam CT, an MRI, a CT scan, a photographic device, a cell phone, a handheld intra-oral photographic device, an intra-oral photographic tray, and a saliva analytic device.

In an embodiment of the method, the health care provider is at least one selected from: a dentist, a physician, a radiologist, and a surgeon. An embodiment of the method further includes prior to scanning, collecting from the subject information for at least one of: insurance, medical history, release consent, and personal identification. In an embodiment of the method, the treatment plan further includes at least one care option.

An embodiment of the method further includes prior to displaying calculating cost for the treatment plan. In an embodiment of the method, the cost further includes an insurance adjusted cost. In an embodiment of the method, the treatment plan further includes at least one of: a location, an appointment time, and a subject education material.

An embodiment of the method further includes prior to sending, selecting a category of the health care providers based on the initial exploratory results. An embodiment of the method further includes prior to sending, uploading the initial exploratory results to a data storage resource.

An aspect of the invention described herein provides a system to connect a subject to at least one health care provider, the system including: at least one digital computing machine, and an exploratory device.

An embodiment of the system further includes a display device. An embodiment of the system further includes a data storage resource selected from a data server, a cloud server, a removable digital data device. An embodiment of the system further includes an Artificial intelligence processor. In an embodiment of the system, the exploratory device is at least one selected from: an x-ray, an intra-oral scanner, cone beam CT, MRI, CT scan, a photographic device, a cell phone, a handheld intra-oral photographic device, an intra-oral photographic tray, and a saliva analytic device.

### BRIEF DESCRIPTION OF DRAWINGS

FIG. 1A and FIG. 1B are pictorial presentations of a flow chart for a subject that performs a self-exploratory exam.

FIG. 2A and FIG. 2B are pictorial presentations of a flow chart for a subject that performs exploratory exam at an exploratory exam location.

### DETAILED DESCRIPTION

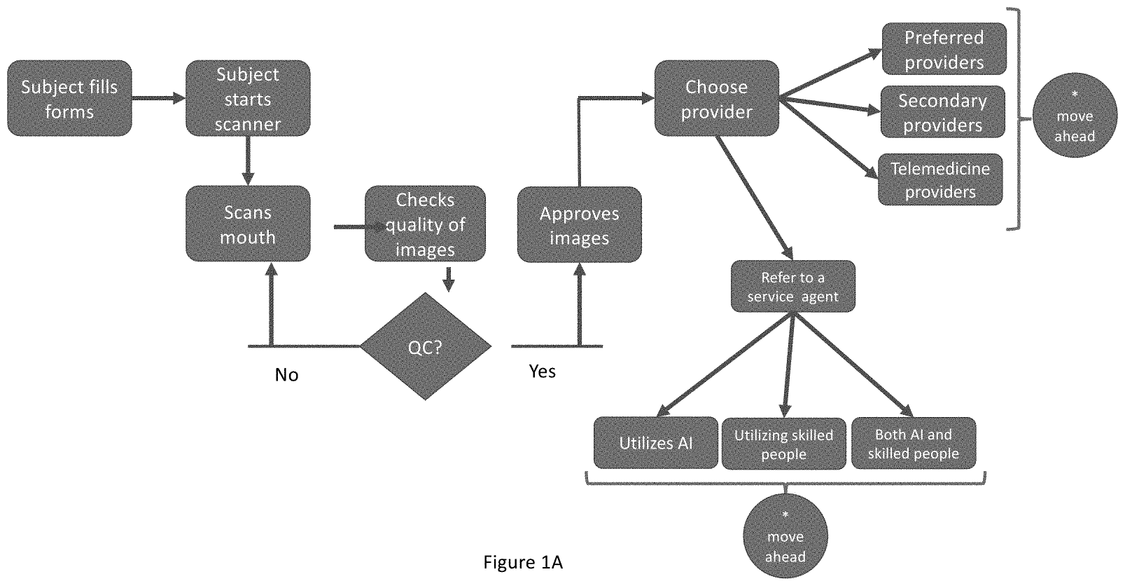

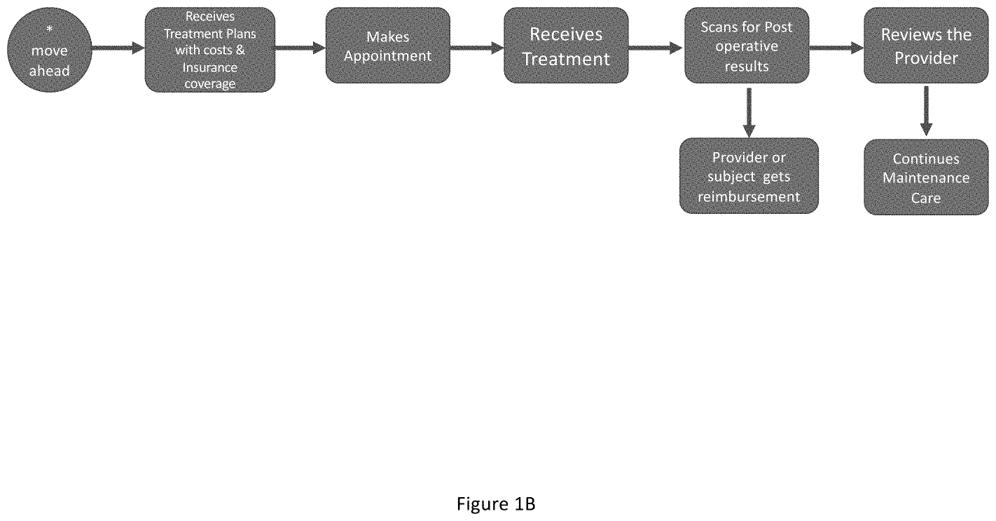

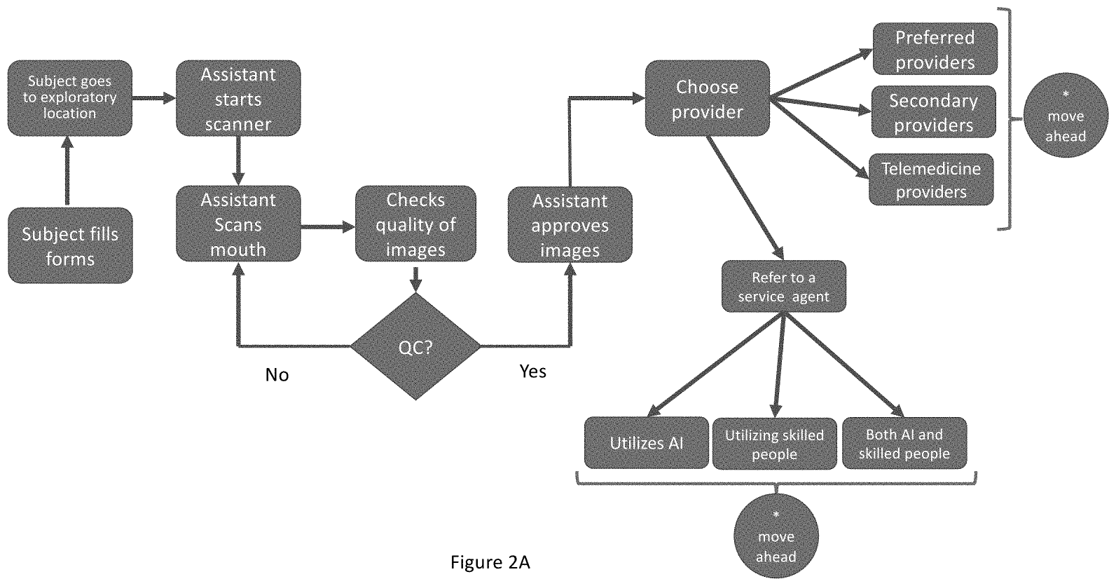

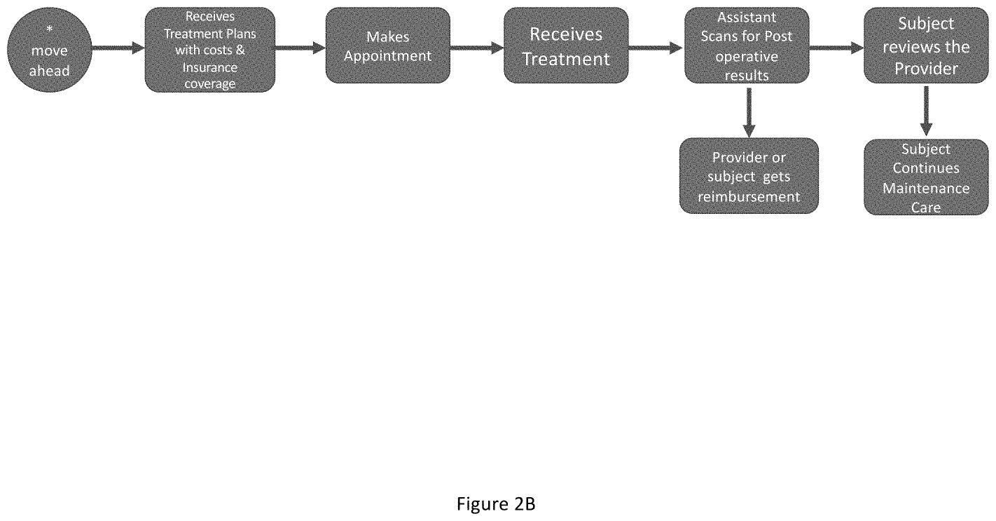

Methods are described herein for a subject to obtain multiple treatment plans including cost for each treatment plan from multiple health care providers from a single appointment. In an embodiment of the method, the subject arrives at the exploratory exam location, an exploratory exam is performed. In alternative embodiments, the subject performs the exploratory exam at home, or at a retirement home, or at assisted living facility. The exploratory exam location is at least one selected from: mobile health clinics, urgent care, emergency care, dental clinic, veterinary office, hospital, veterinary hospital, healthcare camps, travelling clinics, and retail clinics as described in FIG. 2A and FIG. 2B. In some embodiments, the exploratory exam is performed by an authorized assistant. In alternative embodiments, the exploratory exam is performed by the subject themselves as described in FIG. 1A and FIG. 1B. The subject or an authorized representative completes forms including personal information, insurance information, consent, Health Insurance Portability and Accountability Act (HIPAA) and/or medical history.

In some embodiments, the subject, or the authorized representative consents to release the exploratory exam results to at least one of: the subject, insurance provider records, a preferred provider, a non-preferred provider, a secondary provider, a telemedicine service provider, and provider referral service. In some embodiments, the exploratory exam results are saved for health data usage, public health related applications or for data mining service organizations. In some embodiments the subject or the authorized representative completes the forms prior to arriving at the exploratory exam location. In alternative embodiments, the subject completes the forms at exploratory exam location. The forms are either in paper format or in an electronic format. The electronic forms are completed using a computer or a tablet or a mobile device or the exploratory device. The subject accesses the electronic forms by scanning a QR code in some embodiments. The forms are uploaded to a secure server, for example a cloud service or a data storage service. In alternative embodiments, the forms are uploaded on a portable data storage device.

The exploratory exam is performed by the subject, a licensed health care provider such as a physician assistant, a nurse practitioner, a registered nurse, a dental assistant, a physical therapist, a dentist, a physician, a dental student, a medical student, or a mental health professional. The exploratory exam is an initial exam to perform an initial diagnosis or an initial assessment for example, the exploratory exam is a radiation or a non-radiation exam. Examples of non-radiation exam are an intra-oral scan, a photographic device, a cell phone, a handheld intra-oral photographic device, an intra-oral photographic tray, a saliva analytic device, etc. Examples of radiation exam are X-ray, CT, MRI, Cone Beam CT, etc.

Upon completing the exploratory exam, the results are uploaded to a secure server. In alternative embodiments, the exploratory results are assessed and classified based on severity of the subject's health conditions. In some embodiments, the exploratory results are color-coded based on severity. In some embodiments, the classification is performed by Artificial Intelligence (AI). For example, an intra-oral scanner simultaneously records 3D, intraoral color and near Infra-Red (NIRI) images and provides an oral health snapshot for the subject. The snapshot includes presence of caries, missing teeth, teeth alignment, gum disease, etc. Based on the snapshot the subject is classified as green, yellow, and red. Subjects having good oral health and requiring minimum procedures to restore their oral health are classified green. Subjects having moderate quality of oral health and requiring substantial procedures to restore oral health are classified yellow. Subjects having poor quality of oral health and requiring extensive procedures to restore their oral health are classified red.

In some embodiments, the exploratory results are classified based on the specialty of the health care provider, or acceptance of insurance plans, or geographical location or provider rating, or provider availability. For example, provider availability may be based on hours of operation, availability in appointment schedules, or urgency of treatment required. In some embodiments, the exploratory results are classified by Artificial Intelligence or by qualified professionals or by AI and qualified professionals.

The exploratory results and/or the classified subject categories are uploaded to a secure server. The health care providers access the secure server and assess the exploratory results. In alternative embodiments, upon uploading the exploratory results the health care providers or their clinics receive an alert regarding a potential new subject or an existing subject of record.

The health care providers analyze the results and assemble at least one treatment plan for the subject. For example, a dentist prepares a first treatment plan including procedures such as periodontal treatment, restorations and extractions. The dentist may prepare a second treatment plan including procedures such as periodontal treatment, restorations, root-canal treatment and crowns. The dentist may prepare a third treatment plan including procedures such as periodontal treatment, restorations, root-canal treatment and crowns, extractions, and implants, or dentures. The dentist may prepare a fourth treatment plan including procedures such as periodontal treatment, restorations, root-canal treatment and crowns, extractions, implants, or dentures, and orthodontic aligners. The treatment plans are uploaded to the secure server and are accessible by the subject. In alternative embodiments, the subject receives a notification on their phone, the application, or an email alerting them to review the treatment plan. In alternative embodiments, the treatment plan includes cost for each treatment option. The cost may include cost covered by the health insurance and the out-of-pocket costs. In alternative embodiments, the health care providers may also include available financing options in the treatment plan.

The subject chooses a health care provider based on their preference. In alternative embodiments, the subjects may access reviews published for the health care providers, or their health care ratings, or any other satisfaction survey results.

The inventions described herein are the most practical methods. It is recognized, however, that departures may be made within the scope of the invention and that modifications will occur to a person skilled in the art. With respect to the above description then, it is to be realized that the optimum dimensional relationships for the parts of the invention, to include variations in size, materials, shape, form, function, steps, and manner of operation, assembly and use, would be apparent to one skilled in the art, and all equivalent relationships to those illustrated in the drawings and described in the specification are intended to be encompassed by the present inventions.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

### EXAMPLES

### Example 1: Subject Performs Self Exploratory Exam

The subject completes medical history, HIPAA, insurance, consent to release forms on a digital device such as a computer, a smart phone, a tablet, or an exploratory device. The exploratory exam is conducted by the subject or an authorized representative or a family member, a friend, etc. as described in FIG. 1A and FIG. 1B. The exploratory exam is conducted using the camera of a mobile device or a handheld exploratory device such as a portable home dental tracking and checkup device. The subject verifies the quality of exploratory exam results. If the quality is not optimal, the subject repeats the exam. The subject approves and saves the results. The subject may choose to receive the results, send the results to one or more preferred providers or to telemedicine services or to provider referral service. If the subject chooses one or more preferred providers the results are sent to the preferred providers or telemedicine services via electronic email, paper copies, electronic storage devices, or cloud services.

If the subject chooses provider referral service, then the results are uploaded to a cloud service. The subject then chooses at least one parameter selected from: geographical location of the providers, specialty of care required, and insurance acceptance. The provider referral service selects multiple referred providers based on the subject's chosen parameters and sends the exploratory results to the multiple referred providers. The provider referral services select the multiple referred providers using AI or qualified human technicians or both. The multiple referred providers formulate one or more treatment plans including cost and insurance coverage information and send the treatment plan to the subject. The subject then chooses an appropriate provider, makes appointment and receives treatment. The subject may perform a second exploratory exam for post operative results and records. The subject may rate the provider or post a review.

### Example 2: Subject Performs Exploratory Exam at an Exploratory Exam Location

The subject arrives at an exploratory exam location as described in FIG. 2A and FIG. 2B. The subject may complete medical history, HIPAA, insurance, consent to release forms at the exploratory location or prior to arrival. The exploratory exam is conducted by a licensed or qualified provider such as a dental assistant or a dentist or a technician. For example, the dental assistant may use an intra oral image capturing device to take intra-oral pictures of the subject. The provider verifies the quality of exploratory exam results. If the quality is not optimal, the provider repeats the exam. The provider approves and saves the results. The subject may choose to receive the results, send the results to one or more preferred providers or to telemedicine services or to provider referral service. If the subject chooses one or more preferred providers the results are sent to the preferred providers or telemedicine services via electronic email, paper copies, electronic storage devices, or cloud services. If the subject chooses provider referral service, then the results are uploaded to a cloud service.

The subject then chooses at least one parameters selected from: geographical location of the providers, specialty of care required, and insurance acceptance. The provider referral service selects multiple referred providers based on the subject's chosen parameters, and sends the exploratory results to the multiple referred providers. The provider referral services select the multiple referred providers using AI or qualified human technicians or both. The multiple referred providers formulate one or more treatment plans including cost and insurance coverage information and send the treatment plan to the subject. The subject then chooses an appropriate provider, makes appointment, and receives treatment. The subject may perform a second exploratory exam for post-operative results and records. The subject may rate the provider or post a review.

What is claimed is:

1. A method for connecting at least one subject to at least one health care provider, the method comprising:
scanning the subject with an exploratory device to obtain an initial exploratory result;
sending the initial exploratory result to the at least one health care provider for analysis; and
receiving and displaying at least one treatment plan from the at least one health care provider thereby connecting the subject to the health care provider.

2. The method according to claim 1 further comprising prior to sending, identifying at least one health condition in the subject from the initial exploratory result.

3. The method according to claim 2 further comprising after identifying, classifying the subject by severity of the at least one health condition.

4. The method according to claim 1 further comprising after displaying, the subject selecting at least one treatment plan from the at least one health care provider.

5. The method according to claim 1 further comprising choosing at least one of the exploratory device from: an x-ray, an intra-oral scanner, cone beam CT, MRI, CT scan, a photographic device, a cell phone, a handheld intra-oral photographic device, an intra-oral photographic tray, and a saliva analytic device.

6. The method according to claim 1, the health care provider is at least one selected from: a dentist, a physician, a radiologist, and a surgeon.

7. The method according to claim 1 further comprising prior to scanning, collecting from the subject information for at least one of: insurance, medical history, release consent, and personal identification.

8. The method according to claim 1, the treatment plan further comprising at least one care option.

9. The method according to claim 1 further comprising, prior to displaying calculating cost for the treatment plan.

10. The method according to claim 9, the cost further comprises an insurance adjusted cost.

11. The method according to claim 1, the treatment plan further comprising at least one of: a location, an appointment time, and a subject education material.

12. The method according to claim 1 further comprising prior to sending, selecting a category of the health care providers based on the initial exploratory results.

13. The method according to claim 1 further comprising prior to sending, uploading the initial exploratory results to a data storage resource.

14. A system to connect a subject to at least one health care provider, the system comprising: at least one digital computing machine, and an exploratory device.

15. The system according to claim 14 further comprising a display device.

16. The system according to claim 14 further comprising a data storage resource selected from a data server, a cloud server, a removable digital data device.

17. The system according to claim 14 further comprising an Artificial intelligence processor.

18. The system according to claim 14, the exploratory device is at least one selected from: an x-ray, an intra-oral scanner, cone beam CT, Mill, CT scan, a photographic device, a cell phone, a handheld intra-oral photographic device, an intra-oral photographic tray, and a saliva analytic device.
